(19) United States
(12) Patent Application Publication
(10) Pub. No.: US 2005/0261558 A1
(43) Pub. Date: Nov. 24, 2005

(54) Disease risk evaluation tool and method

(76) Inventors: Charles B. Eaton, Sharon, MA (US); David K. Ahern, Tiverton, RI (US)

Correspondence Address: CERMAK & KENEALY LLP, 515 E. BRADDOCK RD, ALEXANDRIA, VA, 22314, US

(21) Appl. No.: 11/106,449

(22) Filed: Apr. 15, 2005

Related U.S. Application Data
(60) Provisional application No. 60/562,312, filed on Apr. 15, 2004.

(51) Int. Cl. G06F 17/60; A61B 5/00

(52) U.S. Cl. 600/300; 705/3

(57) ABSTRACT

A disease risk evaluation and education tool, preferably implemented in logic on a computing device such as a Personal Digital Assistant, permits a user to input patient-specific data relevant to evaluating that patient's risk for a particular disease, e.g., coronary heart disease. The tool's logic calculates the equivalent age of the patient, based on the Framingham data set and on the input data, and presents one or more treatment recommendations.

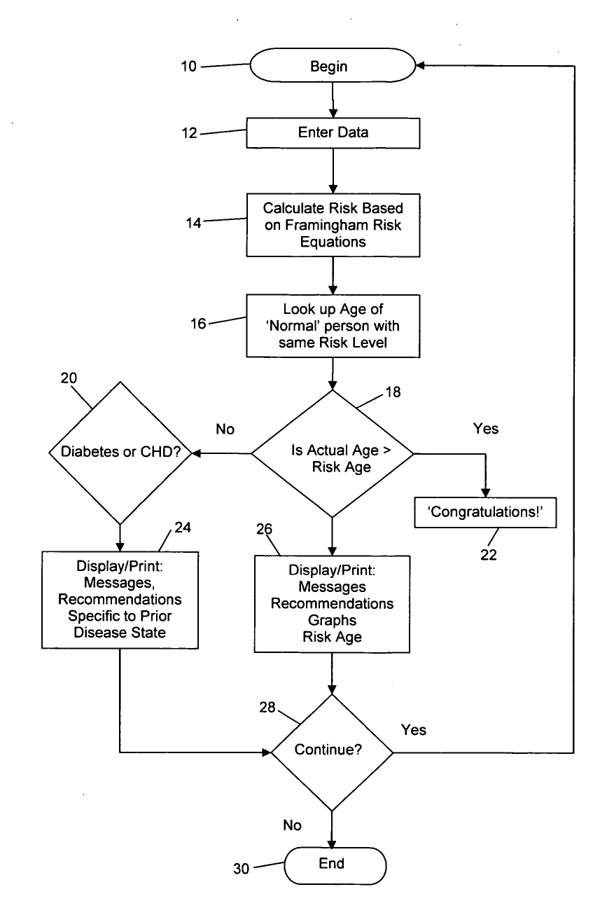

### This application claims priority under 35 U.S.C. § 119 to U.S. provisional application No. 60/562/312, filed 15 Apr. 2004, the entirety of which is incorporated by reference herein.

### STATEMENT AS TO FEDERALLY SPONSORED RESEARCH

This invention was made with U.S. Government support under National Institutes of Health, as part of NHLBI grant RO1HL70804. The federal government has certain rights in the invention.

### BACKGROUND OF THE INVENTION

1. Field of the Invention

The present invention relates to devices, systems, and processes useful as medical patient education and treatment recommendation tools, and more specifically to coronary heart disease (CHD) education and treatment recommendation tools.

2. Brief Description of the Related Art

In 1999, Anderson et al. published a paper that provided a set of equations and look-up tables which would provide a predicted probability of a patient having the onset of CHD within a set period of time. Anderson K M, Wilson P W F, Odell P M, Kannel W B, “An updated coronary risk profile: a statement for health professionals”, Circulation, 1991, 83:357-363 (American Heart Association, Dallas, Tex.) (the entirety of which is incorporated by reference herein). While the Anderson paper provided a very useful update to the classic Framingham Study's risk equations, the form of the equations makes them difficult for a clinician/physician to translate into formulated recommendations for a patient, especially in real or nearly real time. Needless to say, patients themselves may have found the Anderson paper's presentation impenetrable, and the probability results that its equations give to have little meaning to the patient.

The Third Report of the National Cholesterol Education Program Expert Panel on Detection, Evaluation, and Treatment of High Blood Cholesterol in Adults (Adult Treatment Panel III)—Final Report (“ATP III”) (National Institutes of Health, National Heart, Lung, and Blood Institute, NIH pub. no. 02-5215, September 2002) (the entirety of which is incorporated by reference herein) presents recommendations for cholesterol testing and management. While being a definitive document in the area, the use of ATP III to a physician actively consulting with a patient about their cholesterol may be limited by the extensiveness and detail of the report; interpretation of ATP III's recommendations by a particular patient may prove extremely difficult for the same reasons.

Computing devices, including Personal Digital Assistants (PDAs) and other devices, have greatly enhanced the ability of physicians to stay current on medical advances, and to provide treatment recommendations and advice more accurately and quickly to patients. Indeed, many PDA-based tools are currently available for assisting a physician in the examination room by providing portable databases and applications relevant to the physician's practice. There remains a need, however, for a tool that can assist a physician in quickly assembling the data from a patient necessary for the physician to counsel the patient about CHD and lipid disorders, for a tool that presents a patient's CHD and lipid profiles in a form that is simple to interpret, and preferably a tool that can perform both of these function.

### SUMMARY OF THE INVENTION

According to a first aspect of the invention, a disease evaluation tool comprises logic configured to accept data representative of a person's medical condition, and logic configured to calculate the risk of developing coronary heart disease based on said data and on a Framingham data set.

According to another aspect of the present invention, a disease evaluation tool comprises means for accepting data representative of a person's medical condition, and means for calculating the risk of developing coronary heart disease based on said data and on a Framingham data set.

According to yet another aspect of the present invention, a method of evaluating the disease state of a person comprises accepting data representative of a person's medical condition, and calculating the risk of developing coronary heart disease based on said data and on a Framingham data set.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawing, in which the single drawing FIGURE diagrammatically illustrates logic and processes of an exemplary embodiment.

### DETAILED DESCRIPTION OF THE PREFERRED EMBODIMENTS

Referring to the drawing FIGURE, like reference numerals designate identical or corresponding elements throughout the several figures.

A general aspect of the present invention includes a disease risk evaluation tool. While the logic of a tool in accordance with principles of the present invention can be configured as an education and research trial tool for many different mammalian, e.g., human, disease states, one aspect of the present invention is directed specifically at cholesterol education and treatment recommendations. While there are numerous aspects of the present invention, several other aspects include:

1. A simple graphical user interface with a relatively small amount of data entry and clicks, permitting enhanced usage and benefits.

2. Multifunctionality, in that the tool can perform several functions, and modularity, in that some options may be enhanced and activated at a later date.

3. Multiplatform usability, e.g., usable on Palm OS PDA, as well as other OS systems, including general purpose computing devices having an operating system, memory, and an application capable of interpreting and displaying internet and web-based protocols, e.g., html, dhtml, xml, and the like, or other scripting languages in which the logic described herein can be embodied. As will be readily appreciated by those of skill in the art, the principles of the present invention are not limited to PDA-type devices, and the present invention therefore also extends to all computing devices.

A tool in accordance with the present invention performs several functions, including, but not limited to: helping structure a physician's decision making (i.e., a decision support tool); helping educate the physician about treatment guidelines; documenting the patient encounter, with the ability to print information useful for patient care; helping with patient education by enhancing physician-patient communication; and helping patients to carry out a physician's instructions by providing documentation of their cardiovascular risk and reminders of set goals.

A tool in accordance with the present invention preferably includes multiple levels of data collection and/or information presentation, to allow for physician education about aspects of the guidelines and the evidence for the recommendations made. One non-limiting example of a way of developing such levels, hypertext, can allow for multiple levels of exploration.

A tool in accordance with the present invention permits a user, e.g., physician, to tailor the tool using a simple default screen. Optionally, printing of pages can be made available and may optionally provide more information than is on the computing device (e.g., PDA) screen.

Another aspect of the present invention includes that the tool is a decision support tool and not an expert system. The tool presents recommendations based upon the ATP III guidelines, and rationale can be presented for a recommendation and considerations. The quality of the evidence supporting the considerations suggested is made available using the ATP III evidence rating scale, e.g., using hypertext.

Tool Overview

The tool includes logic configured to function in at least the following manner. Those of ordinary skill in the art, being well-versed in implementing scripts and logic on PALM OS, HTML, DHTML, XML and the like, based systems, will immediately appreciate how to implement tools in accordance with the present invention, and therefore code or pseudo-code level details will not be included herein so as not to obscure the principles of the present invention.

In general, data is entered on a data entry screen or page. A decision support tool screen or page for physicians is generated next, and presents lipid diagnosis, current lipid values and goals of therapy, risk stratification, and recommendations & considerations for management. A patient education tool can then be presented in a next screen or page. Print options are optionally made available for both the decision support tool screen or page and the patient education screen or page, the latter of which preferably includes risk factors and risk stratification.

With reference to the single drawing FIGURE, an exemplary logic and processes embodying principles of the present invention are illustrate. After beginning the at 10, the user is prompted to input data 12. The logic then calculates 14 a risk of developing coronary heart disease based on the data and the classic Framingham data set, preferably using equations derived from that data set. The age of a non-diseased or ‘normal’ person is then looked up 16, having the same risk level of disease as calculated. If the actual age of the person is the same or greater than the calculated age, the person is informed 22 that their risk of developing coronary heart disease is average or low for their actual age. If the actual age of the person is less than the age looked up (16), then the indication of diabetes, established coronary heart disease, or both, is queried at 20. If the person does not have either disease, then recommendations, messages, graphs, and the risk age are displayed and/or marked for printing 26. If the person has either disease, then recommendations and messages specific to these disease states are displayed and/or marked for printing 24. Thereafter, the user is prompted 28 to repeat the process, preferably for another person or patient. If the user does not elect to continue, the process ends.

Data Entry Screen or Page

A text message may optionally first be displayed, e.g., describing the permitted use of the tool, copyright notices, and advising that patient care decisions should be made according to the clinician's or physician's judgment.

One or more screens or pages include spaces or fields to enter the following data, either by an embedded script recognition tool (typically a part of a PALM OS device) or a software or attached keyboard. For each field, data validation is performed to determine if the data entered is within a valid range; if it is not, the invalid data is rejected and the particular field is cleared for reentry of the data for that field, optionally with text displayed that the prior data was invalid.

Optionally, the tool logic is configured to record the number of program loads, as well as the number of taps on selected text as follows: “Read First, Goal LDL, Goal Non-HDL.” In addition, the number of check boxes selected (tapped) will be recorded for the following variables: “Diet, Aspirin therapy, Exercise, Decrease BP, D/C Cigarettes, BAS, Statins, Combo, Fibrate, Fish Oil, Niacin, Fibric Acid, CAI, Chest Pain-911, Obese, LFT/CPK, Metabolic Syndrome, Hypercholesterolemia, and TSH.” The tool logic also optionally is configured to provide information about Diet Types (High Blood Pressure, High Cholesterol, Metabolic Syndrome, Weight Loss) and how many times a specific diet was clicked on. Yet further, the tool logic can optionally be configured to provide information about the Survey Counts, by counting how many times the check box was selected for following responses: “Changed Provider Yes, Changed Provider No, Prescribed, Not Prescribed, Change Dose, Confirm Management Plan, Disagree with Management Plan, Changed Patient Yes, Changed Patient No, Agreed Lipid Lowering Tx, More Aggressive Lifestyle Management, Did Not Agree to Recommendation, and No Recommendation Made.”

Before the user, who may be a health care professional, initiates data entry, the logic is configured to provide access to one or more ‘help screens’, which preferably include text, images, links to other resources, or combinations thereof, that describe how to use the various parts of the tool logic, and definitions used throughout the use of the tool, including defining abbreviations.

The tool logic can optionally be configured, according to certain principles of the present invention, so that one or more of the following data types can be entered into the data collection on a single page or screen. By way of example and not of limitation, these data collection screens or pages may be grouped as ‘Lipid Profile’ and ‘Risk Factors’, as will be readily apparent to those of skill in the art.

Total Cholesterol (TC): the tool logic is configured so that if the user taps on the label “TC”, a hypertext message is displayed instructing the user what type of data to enter, e.g., “Total cholesterol in mg/dL, enter by tapping on line”. For data validation, the entered value is preferably greater than 130 mg/dL or less than 500 mg/dL.

High density lipoprotein count (HDL): the tool logic is configured so that if the user taps on the label HDL, a hypertext message is displayed indicating what type of data to enter, e.g., “High Density Lipoprotein cholesterol in mg/dL, enter by tapping on line”. For data validation, the entered value is preferably between 15 mg/dL and 120 mg/dL, inclusive.

Triglycerides (TG): the tool logic is configured so that if the user taps on the label TG, a hypertext message is displayed indicating what type of data to enter, e.g., “Triglycerides in mg/dL, enter by tapping on line”. For data validation, the entered value is preferably between 10 mg/dl and 5000 mg/dl, inclusive.

Low density lipoprotein count (LDL): the tool logic is configured so that if the user taps on the label TG, a hypertext message is displayed indicating what type of data to enter, e.g., “Low Density Lipoprotein cholesterol in mg/dL”. The logic is configured to then calculate the value of [TC-HDL-(TG/5)] unless TG>400 mg/dL, in which case no calculation is made; the result of this calculation is presented in the field as the LDL value. The tool logic is also configured to allow for direct entry of this data.

In order to determine if the patient suffers from a lipid disorder, and the type of lipid disorder, the tool logic is configured to allow entry of the following data values:

Age (if the user taps on the hypertext word “age”, an instruction may optionally be presented, e.g., “age in years, enter by tapping on the line”). For data validation, the entered value is preferably between 20 and 99, inclusive.

Gender: Tap on box (M or F)

Systolic Blood Pressure (BP) (if the user taps on the hypertext word “BP”, an instruction may optionally be presented, e.g., “Systolic blood pressure”: For data validation, the entered value is preferably between 60 and 300, inclusive.

Established Coronary Heart Disease (CHD): Tap on box if the patient has CHD (if the user taps on the hypertext word “CHD”, an instruction may optionally be presented, e.g., “coronary heart disease, MI, aborted MI, unstable angina, coronary artery angioplasty, coronary artery bypass surgery”).

Other clinical atherosclerotic disease (DZ): Tap on box if the patient has a DZ (if the user taps on the hypertext word DZ, an instruction may optionally be presented, e.g., “stroke, TIA, carotid atherosclerosis, aortic aneurysm, peripheral vascular disease, intermittent claudication, femoral-iliac bypass surgery, or angioplasty”).

Diabetes: Tap on box if the patient has diabetes (if the user taps on the hypertext word Diabetes, an instruction may optionally be presented, e.g., “Type I or Type II diabetes mellitus, with or without treatment. FBS>126 mg/dL on two occasions without acute illness”).

ACS: tap on box: Tap on box if the patient has been diagnosed with acute coronary syndrome, non-Q MI, unstable angina, in the past 18 months.

Cigarette use: Tap on box if the patient has smoked (if the user taps on the hypertext word Cigarette, an instruction may optionally be presented, e.g., “1 or more cigarettes smoked per day in the past year”).

Prescription for hypertension (HTN): Tap on box if the patient is currently on drug treatment for hypertension (if the user taps on the hypertext word HTN, an instruction may optionally be presented, e.g., “Presently on drug treatment for hypertension”).

Family history of CHD (Hx of CHD): Tap on box if the patient's family has a history of CHD (if the user taps on the hypertext words Hx of CHD, an instruction may optionally be presented, e.g., “Family history of premature CHD in a first degree relative (in men less than or equal to 55 years old, in women less than or equal to 65 years old)”).

Prescription for lipid reducing therapy (Rx for Lipid): Tap on box if the patient is currently on a drug treatment to lower lipids (if the user taps on the hypertext words Rx for Lipid, an instruction may optionally be presented, e.g., “Presently on lipid lowering drug therapy”). The tool logic also optionally is configured so that, if the patient is on a lipid lowering drug therapy, a pop-up message, page, or screen appears when this box is checked, stating “Check meds presently on, if desire med recommendation”, and provides a number of check boxes for indicating specific lipid lowering medications such as: Statin, Fibrate, Niacin, CAI, BAS, and Fish Oil.

Non-fasting lipid profile: Tap on box if the patient's lipid profile was performed while the patient was not fasting (if the user taps on the hypertext words Non-Fasting lipid profile, an instruction may optionally be presented, e.g., “Limited information will be available using this tool, it has been designed for fasting lipid profiles.” and/or “Non Fasting specimens may lead to . . . Inaccurate LDL Goals and recommendations.”).

The tool logic is also preferably configured to present a loop back to review the data that has been gathered. By way of example and not of limitation, the tool's logic is configured to provide an option to present a full page menu listing one or more of the following options: Lipid Profile Entry, Risk Factors, and Abbreviations. The logic is thus preferably configured to permit the user to tap on ‘Lipid Profile Entry’, and present a text message such as, “Enter the results of patient's lipid profile. Tap on any text for full explanation.” The logic is thus preferably configured to permit the user to tap on ‘Risk Factors’, and present a text message such as “Tap on any text for full explanation of risk factor definition. To select for a patient's risk factor and include it in evaluation and SOAP (Subjective, Objective, Assessment, Plan) note, check the adjacent box. Note: “Fasting” box is checked as a default, but you may uncheck the box to enter a non-fasting lipid profile.” The logic is thus preferably configured to permit the user to tap on ‘Abbreviations’, and present a text message such as “BP: Blood Pressure, CHD: Coronary Heart Disease, Dz: Disease, Fm Hx: Family History, HDL: High-Density Lipoprotein, HTN: Hypertension, ACS: acute coronary syndrome, LDL: Low-Density Lipoprotein, ‘Other clinical atherosclerotic disease’: peripheral arterial disease, abdominal aortic aneurysm, symptomatic carotid artery disease, Rx: Treatment, TC: Total Cholesterol, TG: Triglyceride, NC: Not Calculable.”

Decision Support Tool

The tool's logic is also configured to process the data gathered in the above data gathering screen(s) or page(s), and to present information that can assist the clinician or physician in establishing a treatment course.

Lipid diagnosis (Lipid Dx): The tool's logic is configured to present at least the following options based upon the lipid profile data entered, and includes Mixed Lipid Disorder, LDL-Dominant Lipid Disorder, Isolated Low HDL Cholesterol, Atherogenic Dyslipidemia, Triglyceride-Dominant Lipid Disorder, or Unable To Be Determined (since patient is already on lipid lowering therapy). The logic also preferably lists the ICD-9 Codes for the diagnosis.

LDL and HDL Goals

A LDL goal is determined based upon the risk category of the patient. If the non-fasting box was checked, or if the patient is on a lipid prescription, then the LDL goal is calculated but the tool logic displays the warning “may not be valid”. If a CHD risk equivalent (e.g., established CHD, other clinical atherosclerotic disease, diabetes), or if the 10-Yr Framingham Risk>20%, then the LDL goal is <100 mg/dL. If two or more risk factors (age in men equal to or greater than 45 years, or 55 in women, cigarette use, hypertension, family history, HDL<40 mg/dL), or if the 10-Yr Framingham Risk>=10%, then the LDL goal is <130 mg/dL. If there are zero or one risk factors, then the LDL goal is <160 mg/dL.

The non-HDL goal is 30 mg/dl greater than the LDL goal, but is only calculated if TG>=200 mg/dL, or if non-fasting box is checked.

The following table lists the output from the tool's logic, based on the data previously entered.

| Lipid Profile | Lipid Disorder | ICD-9 |
|---|---|---|
| LDL > goal; HDL < 40 mg/dL or | Mixed Lipid Disorder | (272.4) |
| TG > 200 mg/dL= | | |
| LDL > goal; HDL > 40 mg/dL | LDL-Dominant Lipid | (272.0) |
| and TG < 200 mg/dL = | Disorder | |
| LDL < goal; HDL < 40 mg/dL | Isolated Low HDL | (272.2) |
| and TG < 200 mg/dL = | | |
| LDL < goal; HDL < 40 mg/dL | Atherogenic Dyslipidemia | (272.4) |
| and TG > 200 mg/dL = | | |
| LDL < goal; HDL > 40 mg/dL | Triglyceride-Dominant | (272.1) |
| and TG > 200 mg/dL = | Lipid Disorder | |

When the tool logic displays any of the lipid disorders in the table above, and the “Rx for Lipid” check box has already been checked (indicating that the patient currently is being treated with a lipid-lowering drug, then the tool logic presents a message to the clinician such as “Abnormal Lipid Profile, but can't define lipid disorder due to treatment.”

Similarly, if the non-fasting lipid profile box was checked, then Lipid Dx=“unable to define lipid disorder” or another similar instruction. If none of the combinations listed in the table above are satisfied, then the patient's lipid profile is normal, and the tool logic displays a message such as “NORMAL LIPID PROFILE.”

A tool in accordance with the present invention preferably includes another level of instruction and treatment assistance. The tool logic is therefore optionally further configured so that the clinician/physician can indicate, e.g., click on, the Lipid Dx and the tool logic displays hypertext that explains the lipid diagnosis and gives an evidence-based statement, e.g., from the ATP III guidelines, and a recommendation. The following examples should be considered non-limiting:

LDL dominant lipid disorder: LDL>goal, HDL and Triglycerides were normal.

Evidence statement: ‘Multiple lines of evidence from experimental animals, laboratory investigations, genetic forms of hypercholesterolemia and controlled clinical trials indicate a strong causal relationship of LDL cholesterol and CHD “A1=RCT, Very strong evidence.”’

“Recommendation: LDL cholesterol should continue to be the primary target of cholesterol lowering therapy.”

Mixed lipid disorder: LDL>goal and HDL<40 or Triglycerides>=200.

Evidence statement: Multiple lines of evidence from experimental animals, laboratory investigations, genetic forms of hypercholesterolemia and controlled clinical trials indicate a strong cause relationship of LDL cholesterol and CHD “A1=RCT, Very strong evidence.” “Elevated serum triglycerides are associated with increased risk of CHD. Some species of triglyceride-rich lipoproteins, notably, cholesterol-enriched remnant lipoproteins, promote atherosclerosis and predispose to CHD. C1=Observational and metabolic studies, Very strong evidence.’”

“Recommendation: LDL cholesterol should continue to be the primary target of cholesterol lowering therapy.” “In persons with high triglycerides (>=200 mg/dL), VLDL cholesterol should be combined with LDL cholesterol, yielding non-HDL cholesterol. The latter constitutes atherogenic cholesterol and should be a secondary target of therapy.”’

Isolated low HDL: HDL<40 mg/dL LDL<=goal, TG<200 mg/dL

Evidence statements: “A low HDL cholesterol is strongly and inversely associated with risk for CHD. Higher risk for CHD is multifactorial in causation. Although the inverse relationship between HDL cholesterol and CHD shows no inflection points, any reduction in HDL cholesterol from population means is accompanied by increased risk of CHD. (C1).

“Recommendation: A categorical low HDL cholesterol should be defined as a level of <40 mg/dL in both men and women. A specific HDL-goal level to reach with HDL-raising therapy is not identified. However, non-drug and drug therapies that raise HDL cholesterol are part of management of other lipid disorders and non-lipid risk factors should be encouraged.”

Atherogenic Dyslipidemia: TG>=200 mg/dL and HDL<40 mg/dL

Evidence statements: “Atherogenic dyslipidemia commonly occurs in persons with premature CHD. Strongly associated with abdominal obesity, obesity and physical inactivity. (C1). Weight reduction and increased physical activity will mitigate atherogenic dyslipidemia.(A1) Drugs that modify atherogenic dyslipidemia yield moderate reduction in CHD risk (A2, B2)

“Recommendation: Emphasis of management should be on lifestyle modification including weight control and increased physical activity. Consideration should be given to specific drug therapy, i.e., fibrates or nicotinic acid in higher risk persons.”

Hypertriglyceridemia: TG>=200, HDL>=40

Evidence statement: “Elevated serum triglycerides are associated with increased risk of CHD. Some species of triglyceride-rich lipoproteins, notably, cholesterol-enriched remnant lipoproteins, promote atherosclerosis and predispose to CHD. C1=Observational and metabolic studies, Very strong evidence.”

“Recommendation: Greater emphasis should be placed on elevated triglycerides; first line therapy for elevated serum triglycerides should be therapeutic lifestyle change. In persons with high serum triglycerides, elevated remnant lipoproteins should be reduced in addition to lowering of LDL cholesterol. In persons with high triglycerides (>200 mg/dL), VLDL cholesterol should be combined with LDL cholesterol, yielding non-HDL cholesterol. The latter constitutes atherogenic cholesterol and should be a secondary target of therapy.”

Goals

The tool logic is also configured to determine an LDL goal based upon the risk category of the patient.

If ‘nonfasting’ box checked or if the patient is on Lipid RX, then LDL goal is calculated, and the logic preferably displays a message such as ‘may not be valid’.

A ‘% reduction to reach goal’ is determined based upon the patient's current LDL or non-HDL value and patient's LDL or non-HDL goal as calculated by the following formulae:
((LDL−LDLgoal)/LDL)*100 or ((non-HDL-non-HDLgoal)/non-HDL)*100.

The goal value used in the formula is actually the highest value—for example, if the patient's goal LDL is <100, the LDL goal used in the formula is 100. Preferably, the patient's 10-Yr CHD Risk will also be listed under Goal.

If the risk is very high [(Acute Coronary Syndrome), (Diabetes and Established CHD or other clinical atherosclerotic disease), (Established CHD or other clinical atherosclerotic disease or Diabetes and smoker), (established CHD or other clinical atherosclerotic disease and TG>200 mg/dL, HDL<40 mg/dL and/or non-HDL cholesterol>130 mg/dL)], then LDL goal<70 mg/dL; and non-HDL goal<100 mg/dl; and the % reduction to meet goal is calculated and displayed.

If CHD risk equivalent risk (established CHD, other clinical atherosclerotic disease, diabetes), or if 10-Yr Framingham Risk>20%, then LDL goal is <100 mg/dL, and the % reduction needed to reach goal is calculated and displayed.

In this context, risk factors include: age in men equal to or greater than 45 years, or 55 in women, cigarette use, hypertension, family history of premature CHD (<55 in men, >65 in women), HDL≦40 mg/dL.

If the patient's risk is categorized as ‘High Risk’ (2 or more risk factors and 10-Yr Framingham Risk 10-20% and smoker, or If 2 or more risk factor and 10-Yr Framingham Risk 10-20% and TG≧200 mg/dL and non-HDL≧160 mg/dL, or if 2 or more risk factors and 10-Yr Framingham Risk 10-20% and HDL<40 mg/dL), then LDL goal is <100 mg/dL; % reduction needed to reach goal is calculated and displayed, such as “Patient's 10-Yr CHD Risk is ______%.”

If the patient's risk is categorized as ‘Moderate High’ risk (2 or more risk factors, and if 10-Yr Framingham Risk 10-20% but not a smoker, HDL>40 mg/dL and TG<200 mg/dL or non-HDL<160 mg/dL, then LDL goal is <130 mg/dL, and the % reduction needed to reach goal is calculated and displayed, such as “Patient's 10-Yr CHD Risk is ______%.” Optionally, the logic can display “Consider lowering LDL goal to <100 mg/dL if >=2 fam with +fam hx of CHD, or hs-CRP>3 mg/dL or elev coronary calcium >75th %”.

If the patient's risk is categorized as ‘Moderate’ risk (2 or more risk factors, and if 10-Yr Framingham Risk <10%, then LDL goal is <130 mg/dL, the % reduction needed to reach goal is calculated and displayed, such as “Patient's 10-Yr CHD Risk is ______%”.

If the patient's risk is categorized as ‘low’ Risk (0-1 risk factors), then LDL goal is <160 mg/dL, the % reduction needed to reach goal is calculated and displayed, such as “Patient's 10-Yr CHD Risk is ______%”.

In general, the non-HDL goal is 30 mg/dl greater than LDL goal, but is only calculated if TG>=200 mg/dL.

The tool logic also preferably displays LDL current values, as entered in previously, goals for LDL levels, and recommendations. The patient's LDL Current Values comes from data entry screen, unless the non-fasting profile box is checked, then “not accurate” message is displayed. Otherwise, the LDL level is always displayed, even if it is at or below the LDL goal. Additionally, the patient's non-HDL cholesterol is calculated if the patient's TG>=“200 mg/dl [TC-HDL], or if a non-fasting specimen was collected. The non-HDL level is not displayed if TG<200 mg/dl.

Risk Stratification:

The tool logic is configured to then determine and display one or more of the following risk-related indicators: Framingham Risk; Average Risk; equivalent age of the patient's heart (hereinafter, “Age Indicator”); and Risk Category.

The Framingham 10-Year CHD Risk is determined from the data entered for the patient; this risk scalar, usually given as a probability (0-1.00) or percentage risk, can be either calculated using the Framingham risk calculator using continuous variables, preferably modified to factor in LVH (0,1) and diabetes (0,1),. Since LVH is not a entered variable, the average value of each age-gender category by decade is used in the Framingham risk equations below to calculate the patients specific 10 yr CHD Risk. If only TC and HDL have been entered, or if the patient is on a lipid prescription, or if the non-fasting profile box was checked, then the 10-yr risk is calculated, but “may not be valid” is added to the risk percentage that is displayed.

Framingham Equations

The following equations are used to determine the probability (0.000-1.000) that a patient will have CHD within a time frame. “Smoking” is 1 for yes, 0 for no; “ECG-LVH” (left ventricular hypertrophy measured by electrocardiography) is 1 for definite yes, 0 otherwise; “Diabetes” is 1 for yes, 0 for no (as defined in Anderson et al, supra). “in” is the natural logarithm; “exp(x)” indicates “e”, the natural base, taken the to “x” power.

First determine a according to the following:
α=11.1122−(0.9119×ln(SBP))−(0.2767×Smoking)−(0.7181×ln(TC/HDL))−(0.5865×ECG-LVH)

Then determine m, which is different for men (mₘ) and women (m_{f}), according to the following:
mₘ=α−(1.4792×ln(age))−(0.1759×Diabetes)
m_{f}=α−5.8549+(1.8515×(ln(age/74))²)=(0.3758×Diabetes)

For both genders, determine μ and σ as follows:
μ=4.4181+m_{m/f}
σ=exp(−0.3155−0.2784×m_{m/f})

“t” is the time period for which the probability of CHD is to be determined. While the present invention is not restricted to a determination for any particular time period, t=10 years can be a useful time period to use in developing patient education and recommendations. Selecting t=10, compute u according to the following:
u=((ln(t)−μ)/σ

The predicted probability P of CHD for time period t is given by:
P=1−exp(−e^{u})

The Average Risk (for the same age individual) is determined from a look-up table that incorporates the Framingham risk equation and uses the average gender- and age-specific median values for smoking, LVH, diabetes, total cholesterol, HDL cholesterol, and systolic blood pressure (see, e.g., Tables 5 and 6 in Anderson; Table III. 1-5, 111.1-6 in ATP III). The following table of data can be incorporated into the tool's logic for determining Average Risk.

| Average Risk for Each | | Average Risk for Each | |
|---|---|---|---|
| Age Group (Males) | | Age Group (Females) | |
| Age Group | | Age Group | |
| (years) | Weighted Avg. Risk | (years) | Weighted Avg. Risk |
| <30 | <1% | <30 | <1% |
| 30-33 | 1% | 30-41 | <1% |
| 34-35 | 2% | 42-49 | 1% |
| 36-39 | 3% | 50-51 | 2% |
| 40-42 | 5% | 52-59 | 3% |
| 43-44 | 6% | 60-62 | 4% |
| 45-46 | 7% | 63-67 | 5% |
| 47-49 | 8% | 68-69 | 6% |
| 50-51 | 9% | 70-72 | 7% |
| 52-53 | 10% | 73-74 | 8% |
| 54-55 | 11% | 75-78 | 9% |
| 56-57 | 12% | 79 | 10% |
| 58-59 | 13% | | |
| 60 | 14% | | |
| 61-62 | 15% | | |
| 63-65 | 16% | | |
| 66-69 | 17% | | |
| 70 | 18% | | |
| 71-72 | 19% | | |
| 73-74 | 20% | | |
| 75 | 21% | | |
| 76-78 | 22% | | |
| 79 | 23% | | |

The Age Indicator of the patient (described elsewhere herein in more detail) is determined by locating the average age of a person of the same gender that has the same 10-yr-CHD risk as the patient; this age corresponds to the Age Indicator of the patient. For example, a 55 year-old man may have a 10-year risk of 21% because of various factors, which is the same for a 64 year old healthy man; thus, the 55 year-old has a Age Indicator of 64. For females, if Age <=35 and CHD Risk <=1, then Age Indicator=age. For females >35 years old, and for all males, Age Indicator is calculated based upon 10-Yr Risk as shown in the table below:

| AGE INDICATOR | | AGE INDICATOR | |
|---|---|---|---|
| BASED ON RISK | | BASED ON RISK | |
| (MALES) | | (FEMALES) | |
| 10-Yr CHD Risk | Age Indicator | 10-Yr CHD Risk | Age Indicator |
| <=1% | 31.5 | <1% | 35.5 |
| 2% | 34.5 | 1% | 45.5 |
| 3% | 37.5 | 2% | 50.5 |
| 4% | 39 | 3% | 55.5 |
| 5% | 41 | 4% | 61 |
| 6% | 43.5 | 5% | 65 |
| 7% | 45.5 | 6% | 68.5 |
| 8% | 48 | 7% | 71 |
| 9% | 50.5 | 8% | 73.5 |
| 10% | 52.5 | 9% | 76.5 |
| 11% | 54.5 | 10% | 79 |
| 12% | 56.5 | >10% | 80 |
| 13% | 58.5 | | |
| 14% | 60 | | |
| 15% | 61.5 | | |
| 16% | 64 | | |
| 17% | 67.5 | | |
| 18% | 70 | | |
| 19% | 71.5 | | |
| 20% | 73.5 | | |
| 21% | 75 | | |
| 22% | 77 | | |
| 23% | 79 | | |
| >23% | 80 | | |

The Risk Category, which is the same for fasting and non-fasting specimens, falls into one of the following:
Diabetes and established CHD or other atherosclerotic disease; acute coronary syndrome; smoker and established CHD or other atherosclerotic disease or diabetes; or 2 risk factors, 10-yr Framingham Risk is >20% and TG>200 mg/dL, HDL <40 mg/dL, and non-HDL>130 mg/dL=‘High, High Risk.’
Established CHD, other clinical atherosclerotic disease, diabetes, or if 10-Yr Framingham Risk>20%=‘CHD Equivalent’
2 or more CHD Risk Factors and 10-20% risk and [smoker, or strongly positive family history, or if TG>200, HDL<40 mg/dL and non-HDL>160 mg/dL, or hs-CRP is elevated or if CAC is >75^{th} percentile for age and gender]=‘High Risk’
2 or more CHD Risk Factors and 10-20% risk=‘Moderately High Risk’
2 or more risk factors but <10% 10-Yr-Framingham Risk=‘Moderate Risk’
0-1 risk factors=‘Low Risk’

The tool logic is configured to then display one or more Recommendations, as follows. The following recommendations do not apply, however, if non-fasting lipid profiles were given, or Lipid RX has been checked.

Diet: If patient is not at the LDL or the non-HDL goal, then diet is recommended. The tool logic is configured to display and/or print a set of diet recommendations, with optional levels of information associated by hypertext links. The tool logic is configured to present a link, e.g., labeled “diet” which, when tapped on by the user, provides a set of optional diets. By way of example, the logic can indicate to the user to “Choose Diet Recommended:”, and the following exemplary choices of conditions for which a diet regimen can be provided: High Blood Pressure; High Cholesterol; Metabolic Syndrome; and Weight Loss. If a box is checked for one of these diet choices, a recommended diet will print out on the ‘Physician SOAP page’ under Recommendations and on a ‘Patient Education Handout’ under ‘Benefits of Treatment.’

The tool logic can optionally be configured so that, when one taps a ‘Back’ button on the ‘diet’ section, ‘diet’, under ‘Recommend’, will be automatically checked. If no specific diet is checked, the user will be able to check the Diet box, under Recommend, so that the word diet (without a specific diet) can still be presented on printed pages.

Once a specific diet is checked, it remains checked. To clear a checked Diet, the logic is configured to permit the user to tap on “Clear Entry” on the Diet page, which will clear the data entry for selecting a diet. Preferably, only one diet can be checked in the data set.

Exercise: If the patient is not at their non-HDL goal, or has isolated Low HDL cholesterol, then exercise is recommended. The tool logic is preferably configured so that, if one of established CHD, Diabetes, or other clinical atherosclerotic diagnosis is indicated in the data collection screens or pages, or CHD Risk>20%, then when the word ‘exercise’ is tapped, a hypertext message appears such as “Consider Exercise stress test, prior to prescribing an exercise regimen.”

The tool logic is configured to display a set of exercise recommendations, with optional levels of information associated by hypertext links. With reference to the table below, when the clinician/physician clicks or taps on the hypertext box “Exercise”, then a set of recommendations is displayed, set for printing, or both. In general, however, the tool displays an exercise recommendation, e.g., >200 calories (kCal)/day. The following table is merely illustrative of the exercise recommendations provided by the tool.

EXERCISE
Energy expended per 30 min. of activity (Kcal)

| | | | | |
|---|---|---|---|---|
| Body Weight (lbs.) | 120 | 140 | 180 | 200 |
| Walking (15 min/mile) | 153 | 177 | 228 | 282 |
| Running (8 min/mile) | 306 | 357 | 462 | 564 |
| Bicycling (12 mph) | 207 | 243 | 312 | 384 |
| Swimming (25 yds/min) | 144 | 168 | 216 | 264 |
| Aerobics | 219 | 255 | 327 | 399 |

Additionally, if the ‘smoke’ box has been indicated in the data collection page or screen, then the tool logic is configured to present a message for smoking cessation, e.g., “D/C Cig” will appear under “Recommend”. Tapping on that text, a new screen appears titled “Discontinue Cigarettes” and can include additional text such as: “Cigarette smoking is the most potent cardiovascular risk factor. Giving a clear no smoking message and offering help in quitting will double the quit rate of patients.”

If SBP>140, then the tool logic is configured to present a message to encourage lowering of the patient's blood pressure, e.g., “Dec BP” will appear under “Recommend”. Tapping on that text, a new screen appears titled “Decrease Blood Pressure” and can read: “JNC 7 recommends treating BP to <140/90 mmHg or BP <130/80 in patients with diabetes or chronic renal disease. Majority of patients will need two medications to reach goal. For more information see the JNC 7 Express Report, www.nhlbi.nih.gov/guidelines.”

Optionally, the tool logic is configured so that, if the patient has two risk factors and 10 Yr risk>10% risk, ASA therapy is presented under ‘recommendation’.

Optionally, the tool logic is configured so that, if the user has indicated aspirin treatment, a new screen will be presented titled “Aspirin Treatment” and can present text such as: “Recommend aspirin or antiplatelet treatment. Systematic reviews have found prolonged antiplatelet treatment reduces risk of serious vascular events in people at high risk of ischemic events. One systematic review found that prolonged use of aspirin 75-150 mg is as effective as higher doses.

Medications: The tool logic changes the medication recommendations dependent upon the type of lipid disorder. A drug or drugs will be displayed in a manner to indicate that it is in a preferred status for the particular treatment recommendation. The tool logic is configured to provide hypertext links for all drugs, to display additional information to the clinician/physician. The % LDL lowering to reach the goal, along with expected lowering for each dose of drug, are displayed on the drug hypertext screen, and the cost is optionally also displayed. Risk of treatment, benefits of therapy, relative risk reduction, and reduction in the 10-yr Framingham Risk are further optionally displayed. Further optionally, a recommended follow-up and lab testing are displayed as another layer of information, as well as evidence behind the recommendations can be displayed as yet another layer of information. Another optional aspect of the present invention includes that the tool logic is configured to permit the clinician/physician to indicate (e.g., click, tab, etc.) on a drug name and have it connect to a clinical reference volume (e.g., Epocrates brand reference), an electronic prescribing tool, or both.

The recommendations of medications for each type of lipid disorder are shown below; if there is a most preferred recommendation, it is preferably listed first and in CAPS, with less preferred alternatives listed thereafter.

The tool logic is configured so that, if the user check “On Lipid Lowering Drug Therapy” the first page or screen, then under the page or screen, and printout, for ‘Meds Recommended, the following messages and text appear, based on other data as indicated by the user

If STATIN was checked and:

1) If LDL>goal and TG<200 and HDL>40, then ‘Meds’ include Statin, BAS, CAI, Combo. When one taps on Statin, text is presented such as “Consider increasing dose of Statin, or changing to more potent Statin”; when one taps on BAS, “Consider adding Bile Acid Sequestrant”; when one taps on CAI, “Consider adding Cholesterol Absorption Inhibitor”; when one taps on Combo, “Consider using combination statin plus niacin or statin plus ezetimibe therapy”.

2) If LDL>goal and TG<200 and HDL≦40, then Meds include Statin, BAS, CAI, Niacin. When one taps on Statin, “Consider increasing dose of Statin, or changing to more potent Statin”; when one taps on BAS, “Consider adding Bile Acid Sequestrant”; when one taps on CAI, “Consider adding Cholesterol Absorption Inhibitor”; when one taps on Niacin, “Consider adding Niacin”.

3) If LDL>goal, TG≧200<400 and HDL<40, then Meds include Niacin and Statin. When one taps on Niacin, “Consider adding Niacin; check CPK, LFTs”; when one taps on Statin, “Consider increasing dose of Statin, or changing to more potent Statin”.

4) If LDL>goal, TG≧200<400 and HDL≧40, then Meds include Statin, Niacin, and Fibrate. When one taps on Statin, “Consider increasing dose of Statin, or changing to more potent Statin”; when one taps on Niacin, “Consider adding Niacin; check CPK, LFTs”; when one taps on Fibrate, “Consider adding a Fibrate; check CPK, LFTs”.

5) If LDL>goal, TG≧400, then Meds include Fibrate, Statin, and Niacin. When one taps on Fibrate, “Consider adding a Fibrate; check CPK, LFTs”; when one taps on Statin, “Consider increasing dose of Statin, or changing to more potent Statin”; when one taps on Niacin, “Consider adding Niacin; check CPK, LFTs”.

6) If LDL≦goal, and TG≧400, then Meds include Fibrate and Fish Oil. When one taps Fibrate, “Consider adding a Fibrate; check CPK, LFTs”; when one taps on Fish Oil, “Consider adding Fish Oil”.

7) If LDL≦goal, and TG>200<400, then Meds include Niacin and Fibrate. When one taps on Niacin, “Consider adding Niacin; check CPK, LFTs”; when one taps on Fibrate, “Consider adding a Fibrate; check CPK, LFTs”.

8) If LDL≦goal and TG≦200 and HDL<40, then Meds includes Niacin; when one taps on Niacin, “Consider adding Niacin; check CPK, LFTs”.

9) If LDL≦goal and TG≦200 and HDL≧40, then Meds includes no recommendation.

If FIBRATE was checked and:

1) If LDL>goal and TG<200 and HDL>40, then Meds include Statin, BAS, CAI; when one taps on Statin, “Consider adding Statin; check CPK, LFTs”; when one taps on BAS, “Consider adding Bile Acid Sequestrant as second line therapy”; when one taps on CAI, “Consider adding Cholesterol Absorption Inhibitor as second line therapy”.

2) If LDL>goal and TG<200 and HDL≦40, then Meds include Statin and Niacin. When one taps on Statin, “Consider adding Statin; check CPK, LFTs”; when one taps on Niacin, “Consider adding Niacin as second line therapy; check CPK, LFTS”.

3) If LDL>goal, TG≧200<400, then Meds include Statin, Niacin, and Fibrate. When one taps Statin, “Consider adding Statin; check CPK, LFTs”; when one taps on Niacin, “Consider adding Niacin as second line therapy; check CPK, LFTS”; when one taps on Fibrate, “Consider increasing dose of Fibrate”.

4) If LDL>goal, TG≧400, then Meds include Fish oils, Fibrate, and Statin. When one taps on Fish Oil, “Consider adding Fish Oil”; when one taps on Fibrate, “Consider increasing dose of Fibrate”; when one taps on Statin, “Consider adding Statin; check CPK, LFTS”.

5) If LDL≦goal, and TG>200, then Meds include Fibrate and Fish Oil. When one taps on Fibrate, “° Consider increasing dose of Fibrate”; when one taps on Fish Oil, “Consider adding Fish Oil”.

6) If LDL≦goal and TG≦200 and HDL<40, then Meds include Niacin. When one taps on Niacin, “Consider adding Niacin as second line therapy; check CPK, LFTS”.

7) If LDL≦goal and TG≦200 and HDL≧40, then Meds includes no recommendation.

If NIACIN was checked and:

1) If LDL>goal and TG<200, then Meds include Statin, Niacin, BAS, and CAI; when one taps on each word, the logic is configured to display and/or print messages as indicated above.

2) If LDL>goal, TG≧200<400, then Meds include Statin and Niacin; when one taps on each word, the logic is configured to display and/or print messages as indicated above.

3) If LDL>goal, TG≧400, then Meds include Fibrate, Statin, and Fish Oil.; when one taps on each word, the logic is configured to display and/or print messages as indicated above.

4) If LDL≦goal, and TG≧400, then Meds include Fibrate and Fish Oil; when one taps on each word, the logic is configured to display and/or print messages as indicated above.

5) If LDL≦goal, and TG>200<400, then Meds include Niacin; when one taps on each word, the logic is configured to display and/or print messages as indicated above.

6) If LDL≦goal and TG≦200 and HDL<40, then Meds include Niacin; when one taps on each word, the logic is configured to display and/or print-messages as indicated above.

7) If LDL≦goal and TG≦200 and HDL≧40, then Meds includes no recommendation .

If FISH OIL checked and:

1) If LDL>goal and TG<200 and HDL>40, then Meds include Statin, BAS, CAI; when one taps on each word, the logic is configured to display and/or print messages as indicated above.

2) If LDL>goal and TG<200 and HDL≦40, then Meds include Statin, BAS, CAI, Niacin; when one taps on each word, the logic is configured to display and/or print messages as indicated above.

3) If LDL>goal, TG≧200<400 and HDL<40, then Meds include Niacin and Statin; when one taps on each word, the logic is configured to display and/or print messages as indicated above.

4) If LDL>goal, TG≧200<400 and HDL>40, then Meds include Statin, Niacin, and Fibrate; when one taps on each word, the logic is configured to display and/or print messages as indicated above.

5) If LDL>goal, TG≧400, then Meds include Fibrate and Statin; when one taps on each word, the logic is configured to display and/or print messages as indicated above.

6) If LDL≦goal, and TG≧400, then Meds include Fibrate and Fish Oil; when one taps on each word, the logic is configured to display and/or print messages as indicated above.

7) If LDL≦goal, and TG>200<400, and HDL>40 then Meds include Fish Oil, Niacin, and Fibrate; when one taps on each word, the logic is configured to display and/or print messages as indicated above.

8) If LDL≦goal, and TG>200<400, and HDL≦40 then Meds include Niacin and Fibrate; when one taps on each word, the logic is configured to display and/or print messages as indicated above.

9) If LDL≦goal and TG≦200 and HDL<40, then Meds include Niacin; when one taps on each word, the logic is configured to display and/or print messages as indicated above.

10) If LDL≦goal and TG≦200 and HDL≧40, then Meds includes no recommendation.

If BAS checked and:

1) If LDL>goal and TG<200 and HDL>40, then Meds include Statin; when one taps on each word, the logic is configured to display and/or print messages as indicated above.

2) If LDL>goal and TG<200 and HDL≦40, then Meds include Statin and Niacin; when one taps on each word, the logic is configured to display and/or print messages as indicated above.

3) If LDL>goal, TG≧200<400 and HDL<40, then Meds include Niacin and Statin; when one taps on each word, the logic is configured to display and/or print messages as indicated above.

4) If LDL>goal, TG≧200<400 and HDL≧40, then Meds include Statin, Niacin, and Fibrate; when one taps on each word, the logic is configured to display and/or print messages as indicated above.

5) If LDL>goal, TG≧400, then Meds include Fibrate and Statin; when one taps on each word, the logic is configured to display and/or print messages as indicated above.

6) If LDL≦goal, and TG≧400, then Meds include Fibrate and Fish Oil; when one taps on each word, the logic is configured to display and/or print messages as indicated above.

7) If LDL≦goal, and TG>200<400, then Meds Niacin and Statin; when one taps on each word, the logic is configured to display and/or print messages as indicated above.

8) If LDL≦goal and TG≦200 and HDL<40, then Meds include Niacin; when one taps on each word, the logic is configured to display and/or print messages as indicated above.

9) If LDL≦goal and TG≦200 and HDL≧40, then Meds include no recommendation.

If CAI checked and:

1) If LDL>goal and TG<200 and HDL>40, then Meds include Statin; when one taps on each word, the logic is configured to display and/or print messages as indicated above.

2) If LDL>goal and TG<200 and HDL≦40, then Meds include Statin and Niacin; when one taps on each word, the logic is configured to display and/or print messages as indicated above.

3) If LDL>goal, TG≧200<400 and HDL<40, then Meds include Statin and Niacin; when one taps on each word, the logic is configured to display and/or print messages as indicated above.

4) If LDL>goal, TG≧200<400 and HDL≧40, then Meds include Statin and Niacin; when one taps on each word, the logic is configured to display and/or print messages as indicated above.

5) If LDL>goal, TG≧400, then Meds include Fibrate and Statin; when one taps on each word, the logic is configured to display and/or print messages as indicated above.

6) If LDL≦goal, and TG≧400, then Meds include Fibrate and Fish Oil; when one taps on each word, the logic is configured to display and/or print messages as indicated above.

7) If LDL≦goal, and TG>200<400, then Meds include Niacin and Fibrate; when one taps on each word, the logic is configured to display and/or print messages as indicated above.

8) If LDL≦goal and TG≦200 and HDL<40, then Meds include Niacin; when one taps on each word, the logic is configured to display and/or print messages as indicated above.

9) If LDL≦goal and TG≦200 and HDL≧40, then Meds includes no no recommendation.

If “Combo” is tapped, the logic is configured to produce a pop-up screen titled “Info”, with following exemplary text: “If you are using combination therapy, choose the single agent that has the greatest lipid lowering effect to use this decision support program.”

If MEDS is not checked on the first page/screen, then the medications for each type of lipid disorder are shown below. The order and whether in caps makes a difference; the preferred choice is first and in CAPS.

If LDL dominant lipid disorders: STATINS, Bile Acid Sequestrants, Cholesterol Absorption Inhibitors, or combination therapy is programmed for men and for women greater than 45 years old. If the patient is a woman less than 45: the order should be BILE ACID SEQUESTRANT, Cholesterol Absorption inhibitor, Statins, or Combination Therapy.

If Mixed Lipid Disorders LDL>goal & non-HDL>goal, or HDL<40: Statins, Fibrates derivatives, Niacin combination therapy. If non-HDL is greater than the goal, but LDL<goal, then: Fibric Acid derivative, Niacin or Niacin-Statin combination is indicated.

If Isolated Low HDL: Niacin or Niacin-Statin combination is indicated.

If TG-dominant lipid disorder: If TG>=400, then FIBRATES, Fish Oils is indicated.

If TG>=200 but <400, then: Fibrates, Niacin, Statins, is indicated.

If Atherogenic Dyslipidemia: Statin, Niacin, Fibrates is indicated.

Preferably, when the user taps on ‘Statins’ (text), the logic is configured to present a new page or screen, listing one or more preferred medications. Without being limited to a particular brand or medication, this new page of text may display Atorvastatin (Lipitor), Fluvastatin (Lescol), Lovastatin (Mevacor), Pravastatin (Pravachol), Simvastatin (Zocor), and/or Rosuvastatin (Crestor). Further preferably, the logic is configured so that tapping on each of the names of the medications produces yet another page or screen in which dosage(s), percent anticipated lowered LDL effect, percent anticipated increased HDL effect, contraindications, side effects, a summary of clinical trial results, and/or price per day are presented.

If the patient smokes, then “Smoking Cessation” is added under the recommendations.

If SBP>140, then “Improve BP control” is added under recommendations.

If a CHD risk equivalent (established CHD, diabetes, two risk factors, and >20% risk), then “recommend ASA therapy” is added under recommendations.

Further considerations: The tool logic is optionally further configured to present one or more additional considerations, based on the data entered. Without being limited to any particular additional consideration, the following are exemplary additional considerations that the tool logic can be configured to display to the clinician/physician.

If TG-dominant lipid disorder or atherogenic dyslipidemia, or mixed lipid disorder, or if SBP>130, or if HDL<40 in men (<50 in women), or diabetes mellitus, then display ‘consider evaluate for “central obesity”’, and optionally provide a link to an obesity evaluation tool, e.g., the NHLBI obesity tool.

If TG>150 or SBP>130 or HDL<40 mg/dL in men (or <50 mg/dL in women), but not non-fasting profile, then display ‘evaluate for “Metabolic Syndrome”’, and optionally provide a link to, e.g., Metabolic syndrome section of ATP III.

If CHD equivalent risk or CHD, then display “911 with chest pain” lecture’, and optionally provide a link to, e.g., the National Heart Attack Alert PDA tool or similar tool.

If LDL>190 mg/dl, then display ‘consider genetic form of hypercholesterolemia, screen family members, often requires combination therapy.’

For all patients with any lipid disorder, also display ‘Rule out hypothyroidism.’

For all patients when medications are recommended, display ‘Baseline LFT and CPK’

The tool logic is configured so that checkboxes for each of the items in the foregoing “Consider” section record the physician recommendations, e.g., if the physician wants to institute smoking cessation for the patient, then the physician would check this box.

The tool logic is also preferably configured to assemble the data and recommendations indicated by the clinician/physician and present summary pages and/or send the data to a printer for printing. Preferably, one or more of the following are presented in an ‘Information’ screen or page, and/or sent to the printer, preferably configured to be printed on a single page: Name; Risk factors in a box; Recommendations in a box; and all of the elements described above in the Decision Support Tool.

More specifically, the tool logic preferably generates the ‘Information’ page to include a full page menu listing one or more of the following by tapping on an icon or the like: Lipid Diagnosis, Current Values, Recommendations, Meds Recommended, Considerations, Abbreviations, and Printing. The tool logic is also preferably configured to provide one or more of the following, from the ‘Information’ page.

If the user taps on Lipid Diagnosis, then text=“Tap on Lipid Dx for diagnostic criteria, ICD-9 Code, and additional information”.

If the user taps on Current Values, then text=“Patient's current LDL and non-HDL values are listed. If you tap on Goal, the patient's LDL or non-HDL goal, based upon Risk Stratification of the ATP III Guidelines, is listed. In addition, the percent reduction needed to reach this goal from the current value is calculated. The patient's current 10-Yr CHD risk based upon the Framingham Heart Study is also listed”.

If the user taps on Recommendations, then text=“Tap on text for full explanation of the recommendation (i.e., tap on “Diet” and diet recommendations appear). Check the box to include in your SOAP note (available for printing on Bluetooth enabled PDAs and printers).”

If the user taps on Meds Recommended, then text=“Tap on text for options within the listed drug class. (i.e., tap on “Statins” and detailed information on statins will appear). For each drug, the following information is provided: dosage, cost, side effects, contraindications, and clinical trial results. Check box to include treatment in SOAP note (available for printing on Bluetooth enabled PDAs and printers).”

If the user taps on Considerations, then text=“Tap on text for full explanation of the consideration. Check the box to include in your SOAP note (available for printing on Bluetooth enabled PDAs and printers).”

If the user taps on Abbreviations, then text=“ASA: Aspirin, BAS: Bile Acid Sequestrant, BP: Blood Pressure, CAI: Cholesterol Absorption Inhibitors, CHD: Coronary Heart Disease, Combo: Combination Therapy, CP: Chest Pain, CPK: Creatinine Phosphokinase, Dec: Decrease, Dx: Diagnosis, HDL: High-Density Lipoprotein, LFT: Liver Function Test, LDL: Low-Density Lipoprotein, Metab Syn: Metabolic Syndrome, Rhabdo: Rhabdomylasis, TSH: Thyroid Stimulating Hormone, Tx: Treatment.”

If the user taps on Printing, then text=“Tap on “Print” button to print SOAP note for your records. Select a destination printer and tap OK. SOAP note will include patient's risk factors, current lipid profile, lipid diagnosis, LDL and non-HDL goals. Any recommendations, meds recommended, and/or considerations with the box checked will appear in SOAP note under the plan.”

Optionally, the tool logic is configured to process the data to be output to a printer, and may include one or more of the following, preferably on a single page:

Name ______ (optional)

Subjective: includes age, gender, diagnosis, risk factors

Objective: includes lipid profile and BP

Assessment: includes diagnosis, Age Indicator, LDL goal, percent reduction needed, non-HDL Goal, and 10-Yr CHD Risk

Plan: includes recommendations, Meds, and Considerations.

Patient Education (Activation) Tool

The tool logic is also preferably configured to include a set of displays that can assist the clinician/physician in educating the patient. This set of displays, preferably contained on a single screen of the computing device, only comes up if the lipid profile was fasting, or Lipid Rx are not indicated.

A first page of the Patient Education Tool includes up to five bar graphs, including: Risk Now, Risk of Average Person Your Age (i.e., Risk when all risk factors are at goal), Risk if Cholesterol Under Excellent Control (at goal), Risk when BP at goal, and Risk upon Smoking Cessation (these latter two are presented only if the systolic BP greater than 140, or cigarette smoking checked on data entry screen). The scale for the y-axis of the bar graphs is set to be 0-30%, and are labeled “10-Year Risk of Heart Attack per 100”. From the calculations from the Decision Support Tool, the 10-year risk of CHD and the average risk have already been determined, and are displayed for the first and second bar graphs. The third bar graph is generated by using the LDL goal and raising the HDL between 5%-10%, preferably 10%, above its baseline value in the Framingham risk calculation. The data for the fourth and fifth bar graphs are generated by temporarily setting the BP at the goal, and temporarily setting the smoking value to ‘no’, and re-determining the risk value. The Qualitative Risk category based upon ATP III guidelines, namely Extremely High Risk (>20%), High Risk (2RF & 10-20%), Borderline High Risk (2RF & <10%), and Low Risk (0-1RF), are also presented on the display. The Age Indicator is also optionally displayed for the patient, if all the risk factors have been reduced.

The first bar is labeled “You” at the bottom. When a user taps on the bar, hypertext=“Patient at [Qualitative Risk Category]. For Patient Age [patient age], avg. 10-Yr Fram Risk=[Average risk for patient's age here].” The Qualitative Risk category is based upon ATPIII guidelines: Extremely High Risk (>20%), High Risk (2RF & 10-20%), Borderline High Risk (2RF & <10%), Low Risk (0-1RF).

The second bar is labeled “Chol” at the bottom. When a user taps on the bar, hypertext=“Cholesterol Control, 10-Yr Framingham CHD Risk is ______%*. Age Indicator=______”, wherein each is based upon Cholesterol at Goal and HDL raised 10%, and other risk factors remaining unchanged. The new Framingham CHD Risk and Age Indicator are based upon the patient getting cholesterol levels to goal.

The third bar is labeled “Cig” at the bottom. When a user taps on the bar, hypertext=“Smoking Cessation, 10-Yr Framingham CHD Risk is ______%*. Age Indicator=______”, wherein each is based upon Cholesterol at Goal and HDL raised 10%, and other risk factors remaining unchanged. The new Framingham CHD Risk and Age Indicator are based upon the patient getting cholesterol levels to goal.

The fourth bar is labeled “BP” at the bottom. When as user taps on the bar, hypertext=“Blood Pressure Control, 10-Yr Framingham CHD Risk is ______%* Age Indicator=______”, wherein each is based upon Cholesterol at Goal and HDL raised 10%, and other risk factors remaining unchanged. The new Framingham CHD Risk and Age Indicator are based upon the patient getting BP levels to goal.

The Fifth bar is labeled “All” at the bottom. When a user taps on the bar, hypertext=“All Factors at Goal. 10-Yr Framingham CHD Risk is ______%*. Age Indicator=______”, wherein the new Framingham CHD Risk and Age Indicator based upon patient getting all factors to goal.

Further optionally, the tool's logic is configured so that the patient's current Age Indicator will be displayed in the above graph unless LDL or nonHDL>goal, and Age Indicator<chronological age. If LDL>goal and Age Indicator<chronological age, then treated Age Indicator is given for all risk factors at goal. If a user taps on Age Indicator, the hypertext=“Age Indicator is a novel concept uniformly adopted by patients to understand their CHD risk. Age Indicator is the average age of subject in the Framingham Heart Study that has the same 10-yr risk of CHD as your patient.”

Further optionally, the tool logic is configured so that the patient's current percent risk is displayed at the bottom of the first graph (“______% Risk”).

If a user then taps on % Risk, then the tool logic presents hypertext=“Patient's 10-Yr CHD risk based upon Framingham Heart Study.” If Hypercholesterolemia appears under Consider on Screen 2, then hypertext also preferably reads, “For patients with LDL>190, this risk may be grossly underestimated because this patient likely has a genetic lipid disorder.”

The Y-axis preferably reads “10-Year Risk per 100”. If a user taps on this text, then hypertext =“This is the rate per 100 of hard CHD endpoints which include non-fatal MI, fatal MI, and sudden death.”

Printout for Patient and Physician Record

As mentioned above, the tool logic can be optionally configured to provide a printout of data, recommendations, and other items described herein. Preferably, such a printout is only made available for a fasting lipid profile and a patient not on medications. One or more of the following items can be organized by the tool logic for printing, preferably on a single page: Name, Age, Gender, Cardiac risk factors, BP, Lipid Values, Graphs, Age Indicator Before and after, Lipid Diagnosis, Recommendations (from the items indicated in the Decision Support Tool, and Goals.

By way of example and not of limitation, the tool logic can optionally be configured to provide additional screens or pages for display and/or printing, including Risk factor (Diabetes, CHD, and/or other atherosclerotic disease or ACS), Risk category, and Benefits of Treatment.

For example, if: ACS or established CHD has been checked; or if Diabetes and other atherosclerotic disease has been checked, and the patient not at LDL or non-HDL goal; or if established CHD or other atherosclerotic disease has been checked, and the patient is not at LDL or non-HDL goal; then the tool logic can present a warning such as “Patient is at extremely high risk of recurrent coronary heart disease (Age Indicator=80+)” to the user of the tool, in a Risk Factor and Risk Category screen.

A Benefits of Treatment screen or page can optionally be presented, and has numerous possibilities: Cholesterol at goal, Smoking cessation, and Control systolic BP:

If LDL and non-HDL cholesterol are at goal, then text=“Congratulations, by having your cholesterol at goal you have already reduced your risk by 23-47%”.

If LDL or non-HDL Cholesterol NOT at goal, then text=“Cholesterol at goal will decrease your risk 23-47%”.

If Cigarette Use box is checked, then text=“Smoking Cessation: decrease risk 29-42%”.

If Systolic BP>140, then text=“Control Systolic BP: decrease risk 24-57%”

Further optionally, the tool logic can be configured to provide a screen or page for feedback on the usefulness of the tool when evaluating a patient. By way of non-limiting example, the tool can display the following text with data collection and decision tree:

Changed Provider Behavior? Yes No
If Yes
   I prescribed a lipid-lowering medication
   I did not prescribe a lipid-lowering medication
   I changed the dose of a lipid lowering medication
If No
   Tool confirmed my management plan
   Tool disagreed with my management plan

Changed Patient Behavior? Yes No
If Yes
   Patient agreed to lipid lowering Tx
   Patient agreed to more aggressive lifestyle management
If No
   Patient didn't agree to lipid Tx recommendation
   No recommendations made

As will be readily appreciated by those of skill in the art, each of the data types that are collected using the tool can be assigned a default value; optionally, the tool logic is configured to permit the user to reconfigure those default values in a ‘Preferences’ section presented by the tool logic. By way of non-limiting example, the user may choose which variables appear throughout the program from 10 Yr Risk, Average Risk, Risk Category, Age Indicator CHD, Display Cholesterol at Goal, Display Smoking Cessation, and Display Blood Pressure. All boxes can be checked as the default. Unchecking any box prevents that variable from appearing throughout the program. When Age Indicator CHD is checked the default can remain Age Indicator=80+; if that preference is unchecked, it will appear as Age Indicator=N/A when DM is checked, established CHD, or other clinical atherosclerotic disease, on the data entry screen. Further optionally, the user can select which Recommendations you would like to appear from among Lipid Dx, Non-HDL, Recommendations, Consider. All boxes can be checked as the default. The user can uncheck any box to prevent recommendation from appearing throughout the program.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

1. A disease evaluation tool comprising:
logic configured to accept data representative of a person's medical condition;
logic configured to calculate the risk of developing coronary heart disease based on said data and on a Framingham data set.

2. A disease evaluation tool according to claim 1, further comprising:
logic configured to look up the age of a non-diseased person with the same calculated risk.

3. A disease evaluation tool according to claim 2, wherein said logic configured to look up the age of a non-diseased person comprises a look-up table.

4. A disease evaluation tool according to claim 1, further comprising:
logic configured to display recommendations based on the specific calculated risk.

5. A disease evaluation tool according to claim 4, wherein said recommendations comprises recommendations selected from the group consisting of medication recommendations, dietary recommendations, life style recommendations, and combinations thereof.

6. A disease evaluation tool according to claim 1, wherein said data representative of a person's medical condition comprises data representative of a condition selected from the group consisting of total cholesterol level, HDL level, LDL level, TG level, age, gender, systolic blood pressure, established coronary heart disease, other clinical atherosclerotic disease, diabetes, cigarette use, prescription for hypertension, family history of coronary heart disease, prescription for lipid lowering medication, non-fasting lipid profile, acute coronary syndrome, physical activity, hs-CRP, homocysteine, Lp(a), and combinations thereof.

7. A disease evaluation tool according to claim 1, wherein said logic configured to calculate the risk of developing coronary heart disease based on said data and on a Framingham data set comprises logic configured to calculate a probability P using the following equations:
α=11.1122−(0.0110×ln(SBP))−(0.2767×Smoking)−(0.7181×ln(TC/HDL))−(0.5865×ECG-LVH)
mₘ=α−(1.4792×ln(age))−(0.1759×Diabetes)
m_{f}=α−5.8549+(1.8515×(ln(age/74))²)−(0.3758×Diabetes)
μ=4.4181+m_{m/f}
σ_{m/f}=exp(−b 0.3155−0.2784×m_{m/f})
u_{m/f}=((ln(t)−μ)/σ_{m/f}
P=1−exp(−e^{u});
wherein Smoking=1 when the person smokes;
wherein Diabetes=1 when the person has diabetes;
wherein t is the time period for which the probability of CHD is determined; and
wherein the subscript “m” is for a male person and the subscript “f” is for a female person.

8. A disease evaluation tool according to claim 1, further comprising:
logic configured to display recommendations based only on the person having diabetes, established coronary heart disease, or both.

9. A disease evaluation tool comprising:
means for accepting data representative of a person's medical condition;
means for calculating the risk of developing coronary heart disease based on said data and on a Framingham data set.

10. A disease evaluation tool according to claim 9, further comprising:
means for looking up the age of a non-diseased person with the same calculated risk.

11. A disease evaluation tool according to claim 2, wherein said means for looking up the age of a non-diseased person comprises a look-up table.

12. A disease evaluation tool according to claim 1, further comprising:
means for displaying recommendations based on the specific calculated risk.

13. A disease evaluation tool according to claim 4, wherein said recommendations comprises recommendations selected from the group consisting of medication recommendations, dietary recommendations, life style recommendations, and combinations thereof.

14. A disease evaluation tool according to claim 1, wherein said data representative of a person's medical condition comprises data representative of a condition selected from the group consisting of total cholesterol level, HDL level, LDL level, TG level, age, gender, systolic blood pressure, established coronary heart disease, other clinical atherosclerotic disease, diabetes, cigarette use, prescription for hypertension, family history of coronary heart disease, prescription for lipid lowering medication, non-fasting lipid profile, acute coronary syndrome, and combinations thereof.

15. A disease evaluation tool according to claim 1, wherein said means for calculating the risk of developing coronary heart disease based on said data and on a Framingham data set comprises means for calculating a probability P using the following equations:
α=11.1122−(0.9119×ln(SBP))−(0.2767×Smoking)−(0.7181×ln(TC/HDL))−(0.5865×ECG-LVH)
mₘ=α−(1.4792×ln(age))−(0.1759×Diabetes)
m_{f}=α−5.8549+(1.8515×(ln(age/74))²)−(0.3758×Diabetes)
μ=4.4181+m_{m/f}
σ_{m/f}=exp(−0.3155−0.2784×m_{m/f})
u_{m/f}=((ln(t)−μ)/σ_{m/f}
P=1−exp(−e^{u});
wherein Smoking=1 when the person smokes;
wherein Diabetes=1 when the person has diabetes;
wherein t is the time period for which the probability of CHD is determined; and
wherein the subscript “m” is for a male person and the subscript “f” is for a female person.

16. A disease evaluation tool according to claim 1, further comprising:
means for displaying recommendations based only on the person having diabetes, established coronary heart disease, or both.

17. A method of evaluating the disease state of a person, comprising:
accepting data representative of a person's medical condition; and
calculating the risk of developing coronary heart disease based on said data and on a Framingham data set.

18. A method in accordance with claim 17, further comprising:
looking up the age of a non-diseased person with the same calculated risk.

19. A method in accordance with claim 17, wherein said data representative of a person's medical condition comprises data representative of a condition selected from the group consisting of total cholesterol level, HDL level, LDL level, TG level, age, gender, systolic blood pressure, established coronary heart disease, other clinical atherosclerotic disease, diabetes, cigarette use, prescription for hypertension, family history of coronary heart disease, prescription for lipid lowering medication, non-fasting lipid profile, acute coronary syndrome, physical activity, hs-CRP, homocysteine, Lp(a), and combinations thereof.

20. A method in accordance with claim 17,
wherein said calculating the risk of developing coronary heart disease based on said data and on a Framingham data set comprises calculating a probability P using the following equations:
α=11.1122−(0.9119×ln(SBP))−(0.2767×Smoking) −(0.7181×1n(TC/HDL))−(0.5865×ECG-LVH)
mₘ=α−(1.4792×ln(age))−(0.1759×Diabetes)
m_{f}=α−5.8549+(1.8515×(ln(age/74))²)−(0.3758×Diabetes)
μ=4.4181+m_{m/f}
σ_{m/f}=exp (−0.3155−0.2784×m_{m/f})
u_{m/f}=((ln(t)−μ)/σ_{m/f}
P=1−exp(−e^{u});
wherein Smoking=1 when the person smokes;
wherein Diabetes=1 when the person has diabetes;
wherein t is the time period for which the probability of CHD is determined; and
wherein the subscript “m” is for a male person and the subscript “f” is for a female person.
